(19) United States
(12) Patent Application Publication
(10) Pub. No.: US 2026/0053523 A1
(43) Pub. Date: Feb. 26, 2026

(54) RELEASE INSTRUMENTS, DEVICES, SYSTEMS, AND METHODS OF USE AND ASSEMBLY

(71) Applicant: Rafael Medical Devices, LLC, Newark, NJ (US)

(72) Inventor: Gavriel FEUER, Montclair, NJ (US)

(73) Assignee: Rafael Medical Devices, LLC, Newark, NJ (US)

(21) Appl. No.: 18/810,006

(22) Filed: Aug. 20, 2024

(51) Int. Cl. A61B 17/32; A61B 1/317

(52) U.S. Cl. CPC A61B 17/320016; A61B 1/317

(57) ABSTRACT

Videoscopic arthroscopic instruments, devices, and systems are disclosed. The videoscopic arthroscopic system includes a release instrument including at least one camera sensor and a video unit. In addition, methods of assembling the release instruments are disclosed. Further, surgical methods for using the videoscopic arthroscopic instruments, devices, and systems for minimally invasive tissue release surgery to release tissue in a patient's joints or limbs are disclosed.

### FIELD OF THE INVENTION

The present invention relates generally to general surgery, such as instrumentation for endoscopic surgery. More specifically, but not exclusively, the present invention relates to instrumentation for minimally invasive tissue release surgery to release tissue in a patient's joints or limbs.

### BACKGROUND OF THE INVENTION

As endoscopic tissue release procedures have become more common for various conditions, many of the instruments for the procedures have not allowed for full visualization of the anatomic structures during the procedure. Often a separate camera is inserted into the patient to provide visualization of the surgical procedure. Thus, improved instruments are needed for performing the tissue release procedures while allowing the surgeon to fully visualize the area that is being cut.

### SUMMARY OF THE INVENTION

Aspects of the present invention provide videoscopic arthroscopic instruments, devices, and systems, as well as methods for using and assembling the videoscopic arthroscopic instruments, devices, and systems.

In one aspect, provided herein is a release instrument, including handpiece with a first end and a second end and a cannula with a first end and a second end. The handpiece includes at least one housing extending from the second end of the handpiece towards the first end, a sheath portion extending from the at least one housing to the first end of the handpiece, a translating member moveably coupled to the at least one housing, and a blade member moveably coupled to the translating member. The sheath portion of the handpiece is slidingly received within the cannula and the translating member moves the blade member from an undeployed position to a deployed position.

In another aspect, provided herein is a videoscopic arthroscopic system including a release instrument including a blade and at least one camera sensor, wherein the blade and the at least one camera sensor translate together and a video unit coupled to the release instrument.

In a further aspect, provided herein is a surgical method including: obtaining a videoscopic arthroscopic system and creating an incision in a patient. The method also includes inserting a release instrument into the incision and using an integrated camera of the release instrument to position a cannula of the release instrument adjacent to a tissue to be cut. The method further includes deploying the blade from the release instrument and translating a handpiece of the release instrument within the cannula to translate the blade to cut the tissue to be cut. In addition, the method includes removing the release instrument and completing the procedure.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 is a perspective view of one embodiment of a videoscopic arthroscopic system, in accordance with an aspect of the present disclosure;

FIG. 2 is a first perspective view of a release instrument of the videoscopic arthroscopic system of FIG. 1 in an undeployed position, in accordance with an aspect of the present disclosure;

FIG. 3 is a second perspective view of the release instrument of FIG. 2, in accordance with an aspect of the present disclosure;

FIG. 4 is a top view of the release instrument of FIG. 2, in accordance with an aspect of the present disclosure;

FIG. 5 is a bottom view of the release instrument of FIG. 2, in accordance with an aspect of the present disclosure;

FIG. 6 is a first side view of the release instrument of FIG. 2, in accordance with an aspect of the present disclosure;

FIG. 7 is a second side view of the release instrument of FIG. 2, in accordance with an aspect of the present disclosure;

FIG. 8 is a first end view of the release instrument of FIG. 2, in accordance with an aspect of the present disclosure;

FIG. 9 is a second end view of the release instrument of FIG. 2, in accordance with an aspect of the present disclosure;

FIG. 10 is a first end perspective view of the release instrument of FIG. 2, in accordance with an aspect of the present disclosure;

FIG. 11 is a second end perspective view of the release instrument of FIG. 2, in accordance with an aspect of the present disclosure;

FIG. 12 is a first perspective view of the release instrument of FIG. 2 with the top cover and cannula removed, in accordance with an aspect of the present disclosure;

FIG. 13 is a top view of the release instrument of FIG. 12, in accordance with an aspect of the present disclosure;

FIG. 14 is an exploded, first perspective view of the release instrument of FIG. 2, in accordance with an aspect of the present disclosure;

FIG. 15 is an exploded, second perspective view of the release instrument of FIG. 2, in accordance with an aspect of the present disclosure;

FIG. 16 is an exploded, first side view of the release instrument of FIG. 2, in accordance with an aspect of the present disclosure;

FIG. 17 is an exploded, second side view of the release instrument of FIG. 2, in accordance with an aspect of the present disclosure;

FIG. 18 is an exploded, top view of the release instrument of FIG. 2, in accordance with an aspect of the present disclosure;

FIG. 19 is an exploded, bottom view of the release instrument of FIG. 2, in accordance with an aspect of the present disclosure;

FIG. 20 is a cross-section perspective view of the release instrument of FIG. 2 taken along line 20-20 in FIG. 4, in accordance with an aspect of the present disclosure;

FIG. 21 is a perspective view of the release instrument of FIG. 2 in a deployed position, in accordance with an aspect of the present disclosure; and

FIG. 22 is a side view of the release instrument of FIG. 21, in accordance with an aspect of the present disclosure.

### DETAILED DESCRIPTION FOR CARRYING OUT THE INVENTION

Generally stated, disclosed herein are videoscopic arthroscopic instruments, devices, and systems. Further, a method of assembly and a surgical method for using the videoscopic arthroscopic instruments, devices, and systems are discussed.

In this detailed description and the following claims, the words proximal, distal, anterior, posterior, medial, lateral, superior, and inferior are defined by their standard usage for indicating a particular part of a bone or implant according to the relative disposition of the natural bone or directional terms of reference. For example, “proximal” means the portion of a device or instrument nearest the torso, while “distal” indicates the portion of the device or instrument farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

Similarly, positions or directions may be used herein with reference to anatomical structures or surfaces. For example, as the current devices and methods are described herein with reference to use with the bones and anatomical structures of the hand, the bones of the hand, wrist and lower arm may be used to describe the surfaces, positions, directions or orientations of the devices, instrumentation, and methods. Further, the instruments and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the instruments and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the instruments and methods, and the aspects, components, features and the like thereof, described herein with respect to a right hand may be mirrored so that they likewise function with a left hand and vice versa. Further, the instruments and methods, and the aspects, components, features and the like thereof, disclosed herein are described with respect to the hand for brevity purposes, but it should be understood that the devices and methods may be used with other bones of the body having similar structures, for example the elbow or the lower extremity, and more specifically, with the bones of the ankle, foot, and leg.

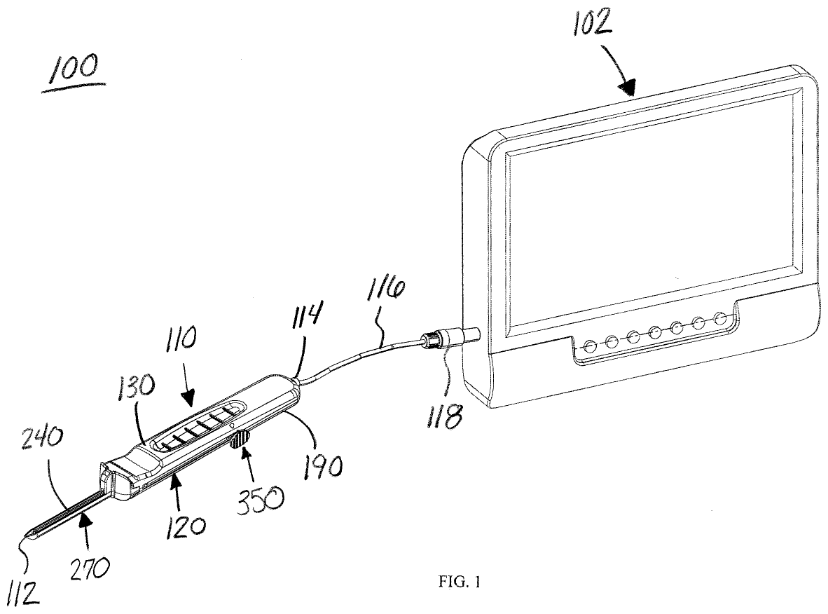

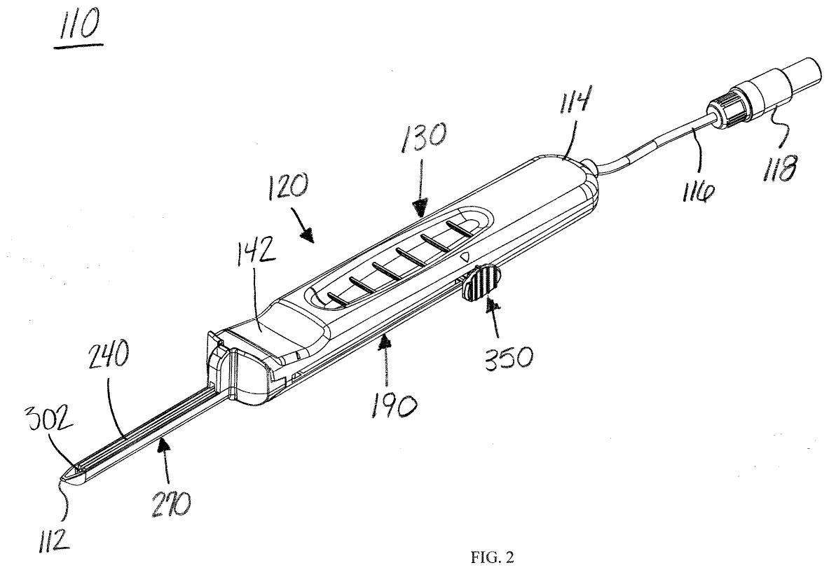

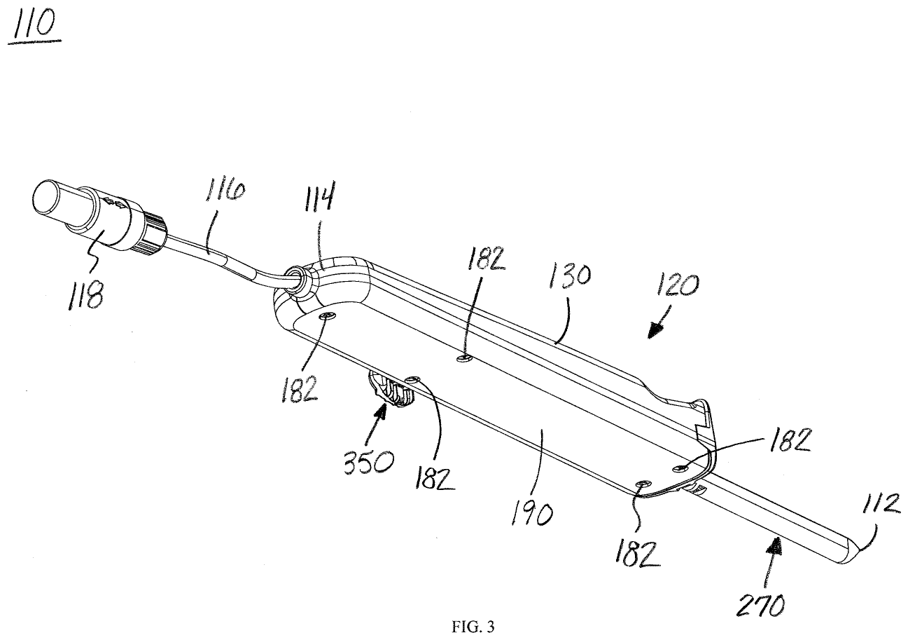

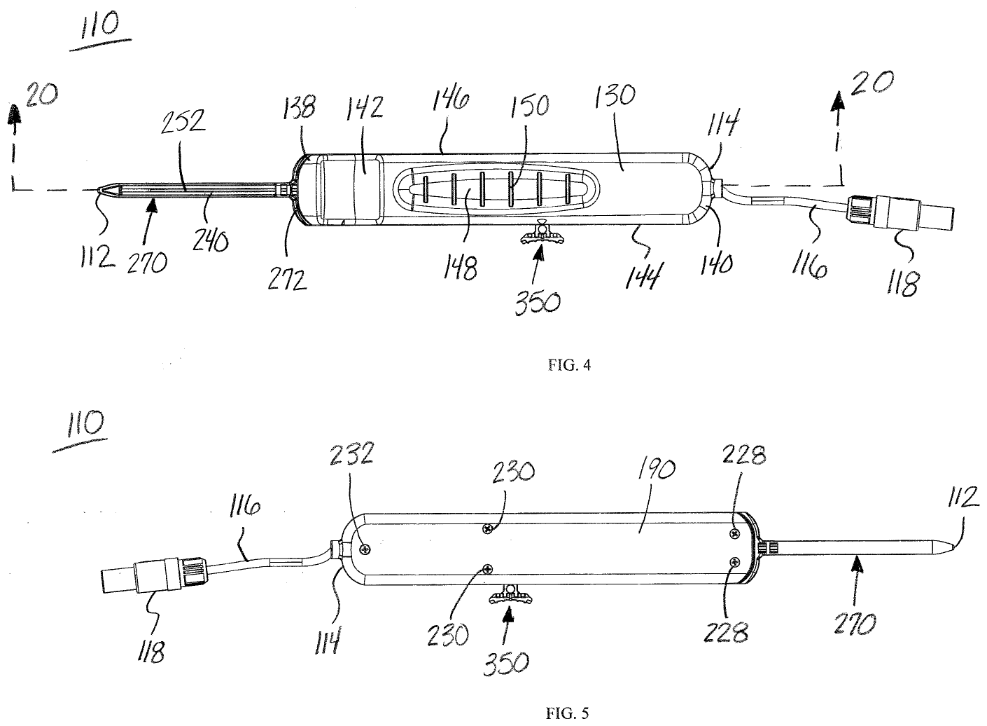

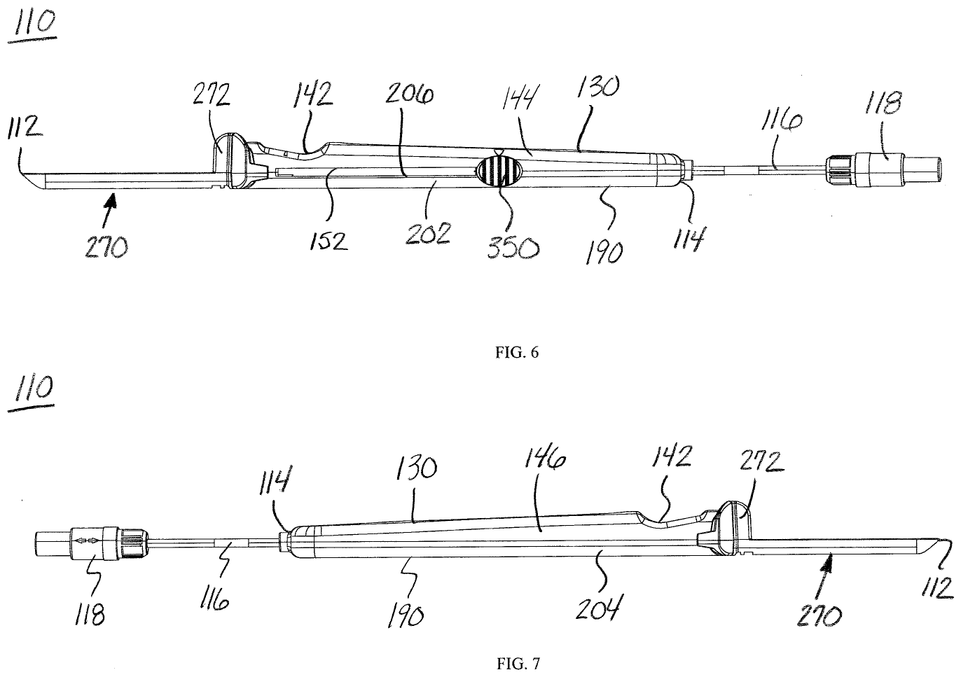

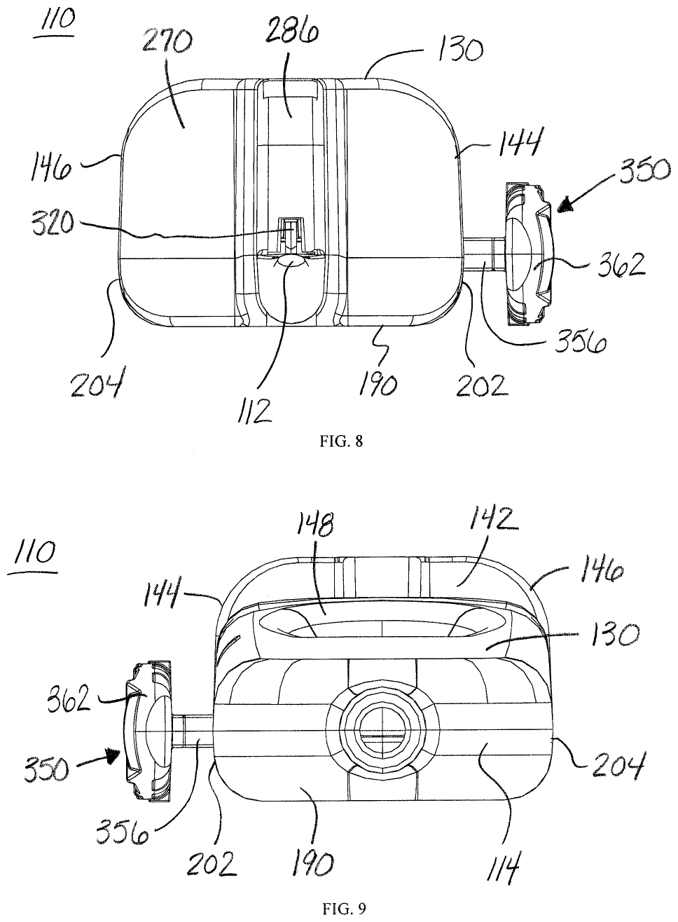

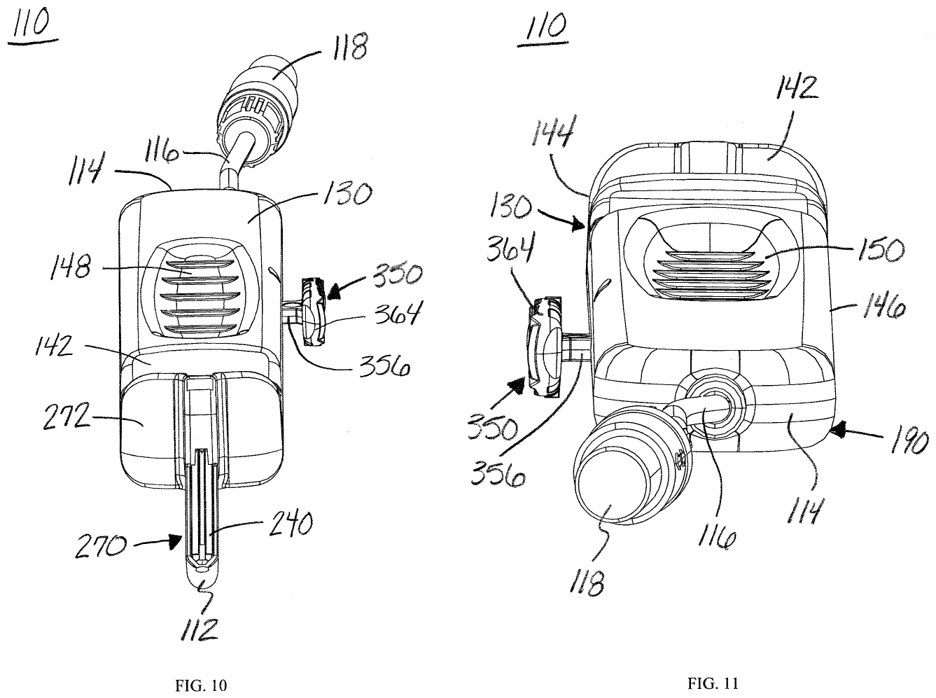

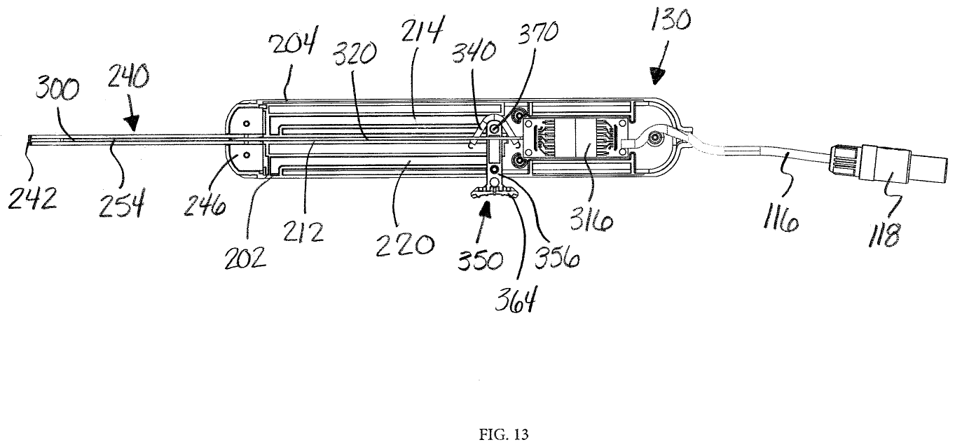

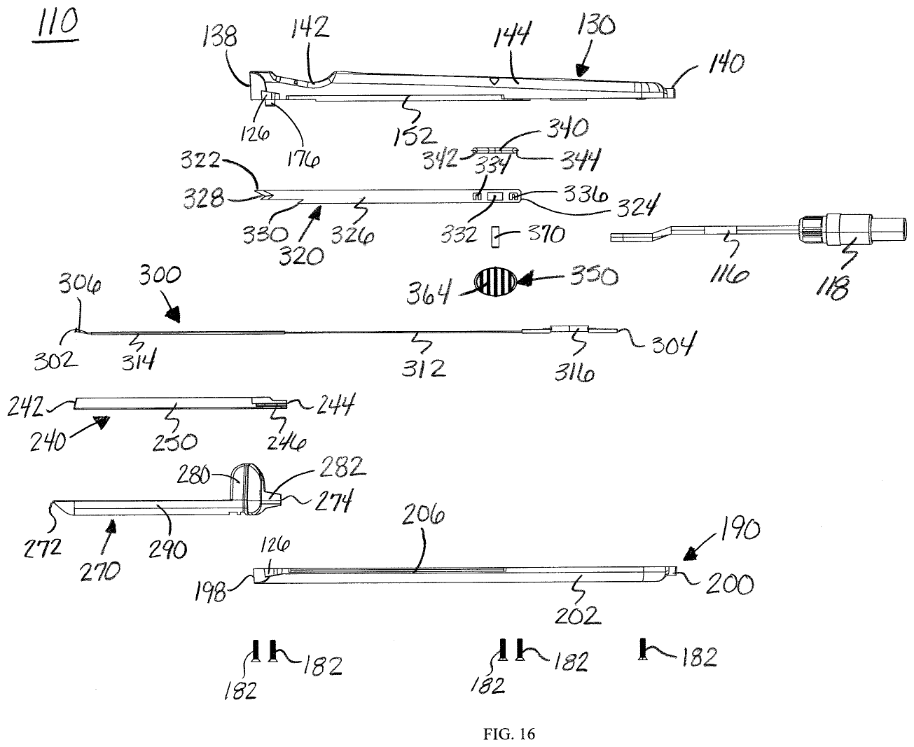

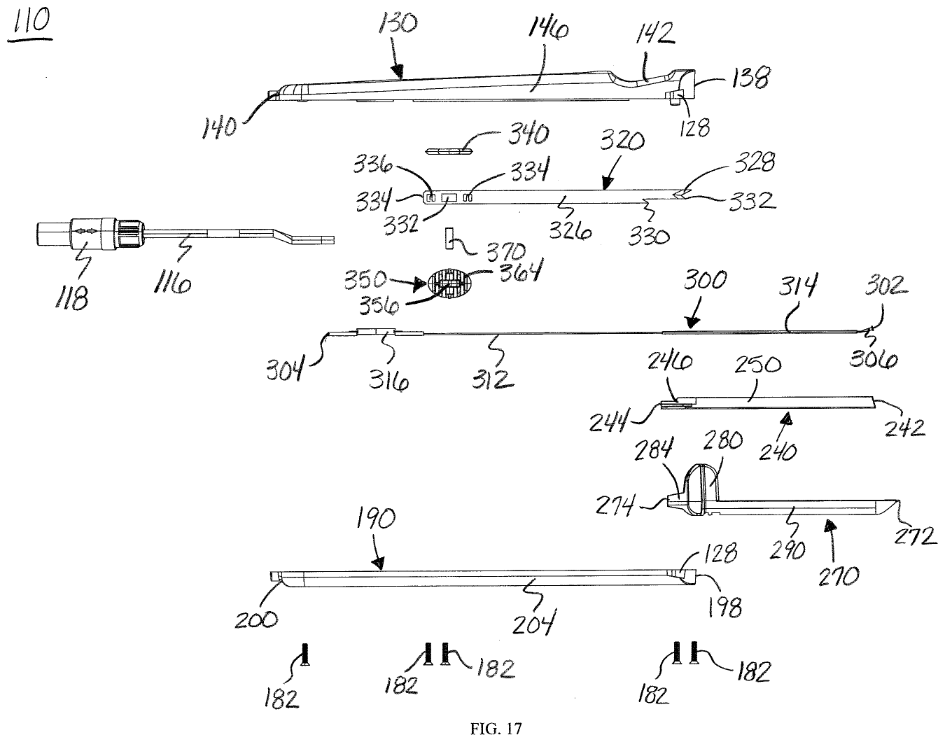

Referring to the drawings, wherein like reference numerals are used to indicate like or analogous components throughout the several views, and with particular reference to FIGS. 1-22, there are illustrated the instruments and devices of the videoscopic arthroscopic system 100. One embodiment of the system 100 may include, for example, a release instrument 110 and a video unit 102. The release instrument 110 may be, for example, a videoscopic, single-use, disposable, arthroscopic instrument for minimally invasive, endoscopic, tissue release surgery in a patient's joints or limbs. The release instrument 110 may also be, for example, for minimally invasive examination, isolation and recession and/or incision of ligament, tendons, or fascia, such as, in carpal tunnel release, plantar fasciotomy, elbow cubital tunnel release, and/or gastrocnemius tenotomy. Specifically, the instrument 110 may be used for tissue release procedures in the wrist, foot, and elbow for cutting tendinous, ligamentous, fibrous tissue, and the like.

With continued reference to FIGS. 1-22, the release instrument 110 may include a first end 112 and a second end 114. The second end 114 of the release instrument 110 may include a cable 116 extending from the release instrument 110 and be coupled to a video unit 102 by a connector 118. The second end 114 may also include a cable attachment member or strain relief member (not shown) extending from the second end 114 of the release instrument 110 with the cable 116 passing therethrough. The video unit 102 may be, for example, a video display, such as a tablet, video processing tablet, video screen, television, and the like, or a video processor that may be separate from or integral with a video display.

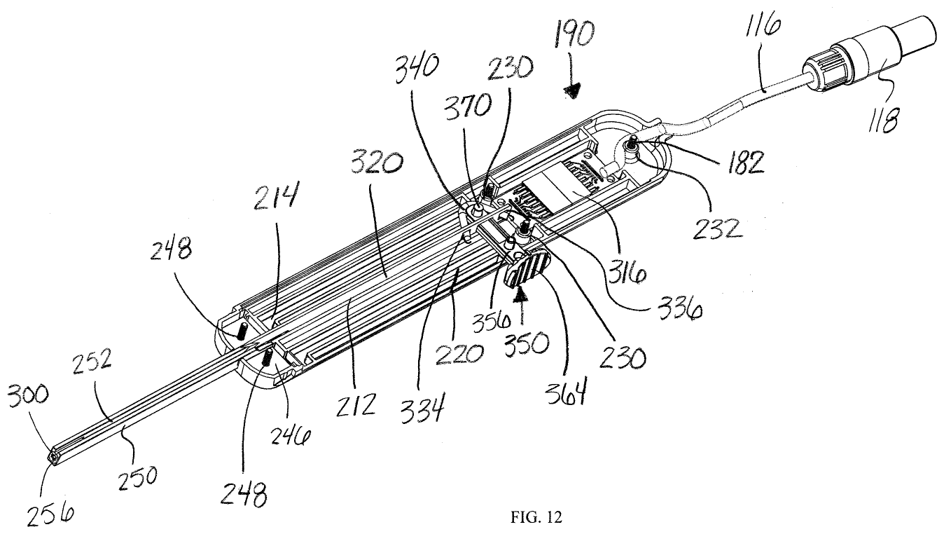

As shown in FIGS. 12-20, the instrument 110 may include a handpiece or housing 120 and a cannula 270. The handpiece 120 may include a first or top housing 130 and a second or bottom housing 190 coupled together to form the handpiece 120. The handpiece 120 may have, for example, a rectangular or cuboid shape. The first housing 130 may include an exterior surface 132 and an interior surface 134. The first housing 130 also includes a handle portion 136 extending from the second end 114 of the instrument 110 towards the first end 112. The handle portion 136 includes a first end 138 and a second end 140. The first housing 130 may be, for example, a singular integral or monolithic piece (i.e., of one-piece construction), or may be formed from a plurality of components that are coupled (i.e., rigidly coupled) together to form the first housing 130. The second housing 190 may include an exterior surface 192 and an interior surface 194. The second housing 190 also includes a handle portion 196 extending from the second end 114 of the instrument 110 towards the first end 112. The handle portion 196 includes a first end 198 and a second end 200. The second housing 190 may be, for example, a singular integral or monolithic piece (i.e., of one-piece construction), or may be formed from a plurality of components that are coupled (i.e., rigidly coupled) together to form second housing 190. The handpiece 120 may also include a sheath portion, camera housing, or blade holder 240 at the first end 138 of the first housing 130 and the first end 198 of the second housing 190. The sheath portion 240 may be, for example, coupled to at least one of the first housing 130 or the second housing 190. The sheath portion 240, first housing 130, and second housing 190 may be, for example, a singular integral or monolithic piece (i.e., of one-piece construction), or may be formed from a plurality of components that are coupled (i.e., rigidly coupled) together to form a coupled handpiece 120.

The exterior surface 132 may include a groove 142 inset into the top surface of the first housing 130 near a first end 138 of the handle portion 136. The groove 142 may extend, for example, between a first side 144 and a second side 146 and may be configured or sized and shaped to receive a user's finger. The exterior surface 132 may also include a recessed region 148 inset into the top surface of the first housing 130 between the groove 142 and the second end 140 of the handle portion 136. The recessed region 148 may have, for example, an oval shape or other shape to fit between the first side 144 and the second side 146. In addition, the recessed region 148 may include, for example, a plurality of protrusions 150 extending within the recessed region 148 in a direction between the first side 144 and the second side 146. The first housing 130 may further include an opening 152 extending from the exterior surface 132 of the first housing 130 into the interior surface 134 from a bottom surface of the first housing 130 towards a top surface. The opening 152 may extend into the first side 144 of the first housing 130. A switch 350 may be positioned to slidingly engage the opening 152 along the length of the first housing 130 between the first end 138 and the second end 140. The first housing 130 may also include a blade passageway 154 extending from the exterior surface 132 into the interior surface 134 from a bottom surface of the first housing 130 towards a top surface. The blade slot 154 may be positioned at the first end 138 of the handle portion 136. The blade slot 154 may be, for example, configured or sized and shaped to slidingly receive a blade or cutting element 320. Additionally, the first housing 130 may include a cable passageway 156 extending from the exterior surface 132 into the interior surface 134 from a bottom surface of the first housing 130 towards a top surface. The cable passageway 156 may be positioned at the second end 140 of the handle portion 136. The cable passageway 156 may be, for example, configured or sized and shaped to receive a portion of the cable 116 as it passes from the interior to the exterior of the handpiece 120.

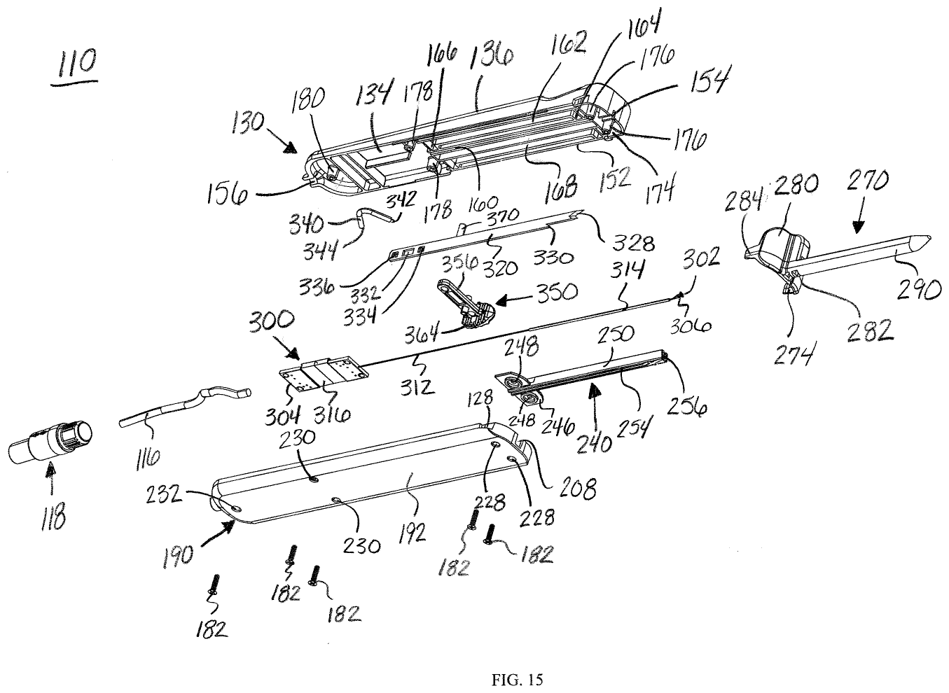

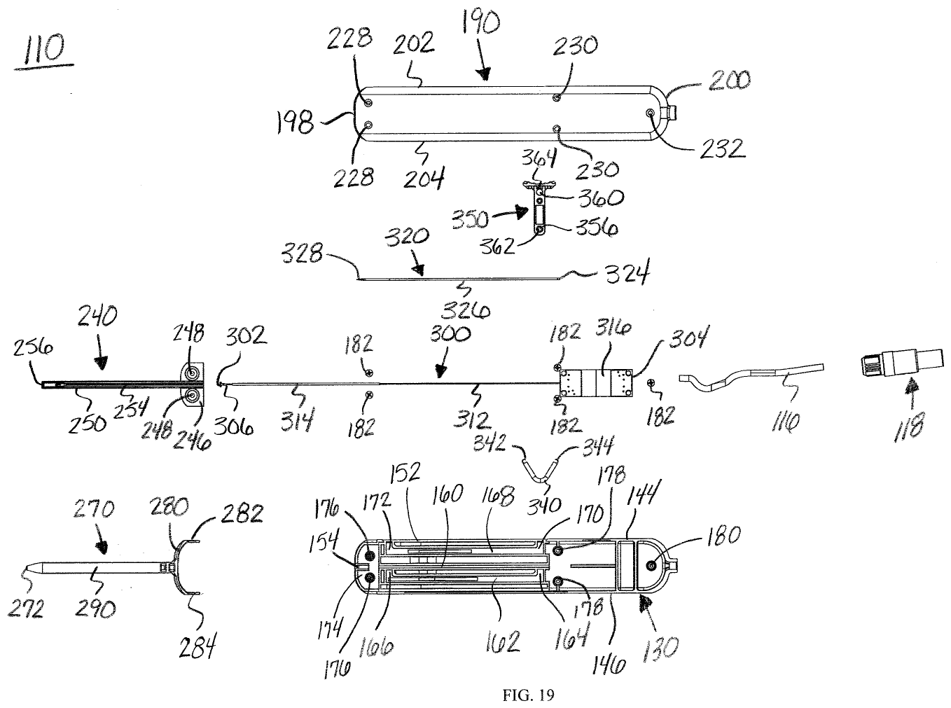

As shown in FIGS. 15 and 19, the interior surface 134 of the first housing 130 may include a plurality of support structures or frames forming a plurality of channels 160, 162, 168.

The plurality of channels 160, 162, 168 may include a first channel 160, a second channel 162, and a third channel 168. The first channel or blade channel 160 may be positioned near a middle of the interior surface 134 between the first side 144 and the second side 146. The blade channel 160 may be, for example, configured or sized and shaped to slidingly receive a blade 320. The blade channel 160 may extend from a position near the first end 138 of the first housing 130 toward the second end 140. The second channel or pin channel 162 may be positioned between the blade channel 160 and the second side 146 of the first housing 130. The pin channel 162 may include a first extension 164 at a first end of the pin channel 162 and a second extension 166 at a second end of the pin channel 162. The extensions 164, 166 extend from the pin channel 162 towards the first side 144 of the first housing 130. The pin channel 162 may be, for example, configured or sized and shaped to slidingly receive a pin 370 of the switch 350. The pin channel 162 may extend from a position near the first end 138 of the first housing 130 toward the second end 140. The third channel or protrusion channel 168 may be positioned between the blade channel 160 and the first side 144 of the first housing 130. The protrusion channel 168 may include a first extension 170 at a first end of the protrusion channel 168 and a second extension 172 at a second end of the protrusion channel 168. The extensions 170, 172 extend from the protrusion channel 168 towards the first side 144 of the first housing 130. The protrusion channel 168 may be, for example, configured or sized and shaped to slidingly receive a protrusion 358, 360 of the switch 350. The protrusion channel 168 may extend from a position near the first end 138 of the first housing 130 toward the second end 140.

With continued reference to FIGS. 15 and 19, the interior surface 134 also includes a recess 174 positioned at a first end 138 of the first housing 130. The recess 174 may be, for example, configured or sized and shaped to receive a portion of the sheath portion 240. The interior surface 134 may also include a plurality of fastener openings 176, 178, 180. The at least one fastener opening 176 may extend through the first housing 130 through the recess 174. The at least one fastener opening 176 may be, for example, two fastener openings 176. A first fastener opening 176 may be positioned between the first side 144 of the first housing 130 and the blade slot 154. A second fastener opening 176 may be positioned between the second side 146 of the first housing 130 and the blade slot 154. The at least one fastener opening 178 may be, for example, two fastener openings 178 positioned between the plurality of channels 160, 162, 168 and a second end 140. A first fastener opening 178 may be positioned between the first side 144 of the first housing 130 and the blade channel 160. A second fastener opening 178 may be positioned between the second side 146 of the first housing 130 and the blade channel 160. The at least one fastener opening 180 may be, for example, one fastener opening 180. The fastener opening 180 may be positioned between the first side 144 and the second side 146 of the first housing 130. Each fastener opening 176, 178, 180 may be, for example, configured or sized and shaped to receive a fastener 182.

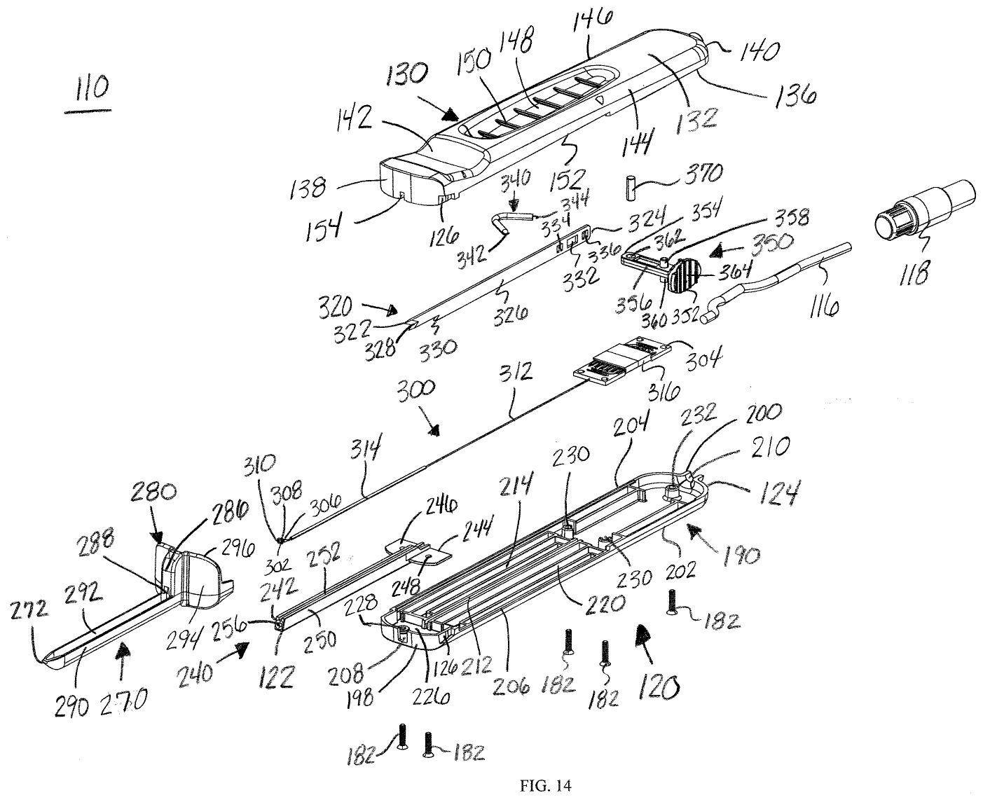

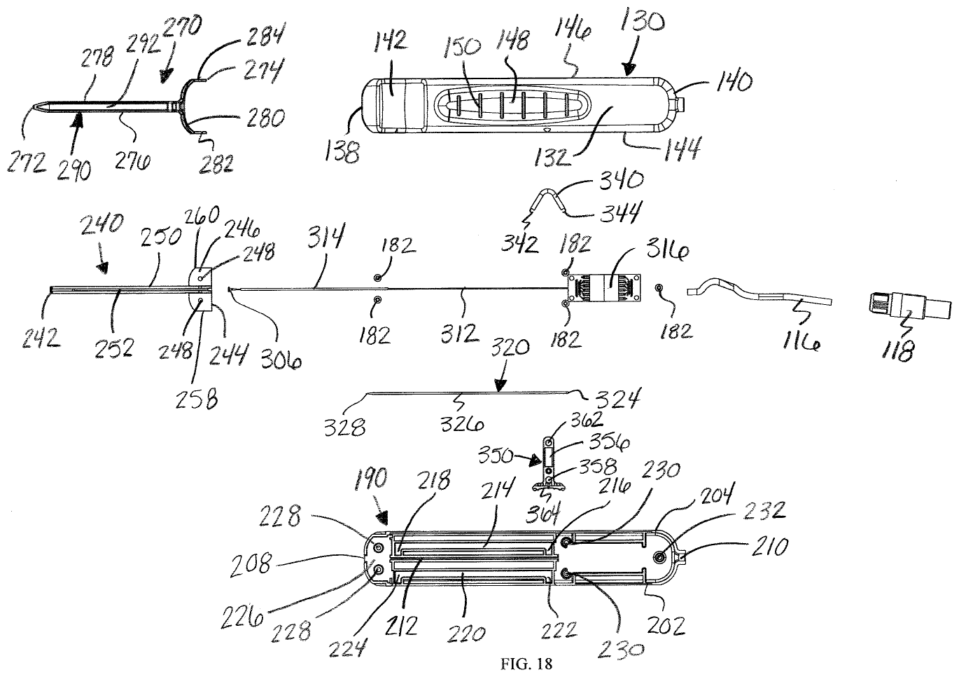

Referring now to FIGS. 14 and 18, the second housing 190 may include an opening 206 extending from the exterior surface 192 of the second housing 190 into the interior surface 194 from a top surface of the second housing 190 towards a bottom surface. The opening 206 may extend into the first side 202 of the second housing 190. The switch 350 may be positioned to slidingly engage the opening 206 along the length of the second housing 190 between the first end 198 and the second end 200. The second housing 190 may also include a blade holder slot 208 extending from the exterior surface 192 into the interior surface 194 from a top surface of the second housing 190 towards a bottom surface. The blade holder slot 208 may be positioned at the first end 198 of the handle portion 196. The blade holder slot 208 may be, for example, configured or sized and shaped to receive a portion of the blade holder 240. Additionally, the second housing 190 may include a cable passageway 210 extending from the exterior surface 192 into the interior surface 194 from a top surface of the second housing 190 towards a bottom surface. The cable passageway 210 may be positioned at the second end 200 of the handle portion 196. The cable passageway 210 may be, for example, configured or sized and shaped to receive a portion of the cable 116 as it passes from the interior to the exterior of the handpiece 120.

With continued reference to FIGS. 14 and 18, the interior surface 194 of the second housing 190 may include a plurality of support structures or frames forming a plurality of channels 212, 214, 220. The plurality of channels 212, 214, 220 may include a first channel 212, a second channel 214, and a third channel 220. The first channel or blade channel 212 may be positioned near a middle of the interior surface 194 between the first side 202 and the second side 204. The blade channel 212 may be, for example, configured or sized and shaped to slidingly receive a blade 320. The blade channel 212 may extend from a position near the first end 198 of the second housing 190 toward the second end 200. The blade channel 212 of the second housing 190 may be aligned with the blade channel 160 of the first housing 130 when the first housing 130 is coupled with the second housing 190. The second channel or pin channel 214 may be positioned between the blade channel 212 and the second side 204 of the second housing 190. The pin channel 214 may include a first extension 216 at a first end of the pin channel 214 and a second extension 218 at a second end of the pin channel 214. The extensions 216, 218 extend from the pin channel 214 towards the first side 202 of the second housing 190. The pin channel 214 may be, for example, configured or sized and shaped to slidingly receive a pin 370 of the switch 350. The pin channel 214 may extend from a position near the first end 198 of the second housing 190 toward the second end 200. The pin channel 214 of the second housing 190 may be aligned with the pin channel 162 of the first housing 130 when the first housing 130 is coupled with the second housing 190. The third channel or protrusion channel 220 may be positioned between the blade channel 212 and the first side 202 of the second housing 190. The protrusion channel 220 may include a first extension 222 at a first end of the protrusion channel 220 and a second extension 224 at a second end of the protrusion channel 220. The extensions 222, 224 extend from the protrusion channel 220 towards the first side 202 of the second housing 190. The protrusion channel 220 may be, for example, configured or sized and shaped to slidingly receive a protrusion 358, 360 of the switch 350. The protrusion channel 220 may extend from a position near the first end 198 of the second housing 190 toward the second end 200. The protrusion channel 220 of the second housing 190 may be aligned with the protrusion channel 168 of the first housing 130 when the first housing 130 is coupled with the second housing 190.

The interior surface 194 of the second housing 190 also includes a recess 226 positioned at a first end 198 of the second housing 190. The recess 226 may be, for example, configured or sized and shaped to receive a portion of the sheath portion 240. The interior surface 194 may also include a plurality of fastener openings 228, 230, 232. The at least one fastener opening 228 may extend through the second housing 190 through the recess 226. The at least one fastener opening 228 may be, for example, two fastener openings 228. A first fastener opening 228 may be positioned between the first side 202 of the second housing 190 and the blade holder slot 208. A second fastener opening 228 may be positioned between the second side 204 of the second housing 190 and the blade holder slot 208. The fastener openings 228 may be aligned with the fastener openings 176 when the first housing 130 is coupled with the second housing 190. The at least one fastener opening 230 may be, for example, two fastener openings 230 positioned between the plurality of channels 212, 214, 220 and a second end 200. A first fastener opening 230 may be positioned between the first side 202 of the second housing 190 and the blade channel 212. A second fastener opening 230 may be positioned between the second side 204 of the second housing 190 and the blade channel 212. The fastener openings 230 may be aligned with the fastener openings 178 when the first housing 130 is coupled with the second housing 190. The at least one fastener opening 232 may be, for example, one fastener opening 232. The fastener opening 232 may be positioned between the first side 202 and the second side 204 of the second housing 190.

Each fastener opening 228, 230, 232 may be, for example, configured or sized and shaped to receive a fastener 182. The fastener opening 232 may be aligned with the fastener opening 180 when the first housing 130 is coupled with the second housing 190.

With continued reference to FIGS. 12-19, the sheath portion 240 includes a first end 242 and a second end 244. The sheath portion 240 includes a base 246 at a second end 244 and an arm 250 extending through a midpoint of the base 246 to the first end 242. The base 246 may have a width larger than the arm 250. In addition, the arm 250 may have, for example, a length longer than the length of the base 246. The base 246 may include at least one opening 248 extending through the base 246 from a top surface to a bottom surface. The at least one opening 248 may include, for example, a first opening 248 positioned between the arm 250 and the first side 258 of the sheath portion 240 and a second opening 248 positioned between the arm 250 and the second side 260 of the sheath portion 240. The arm 250 may include a blade slot 252 extending from the first end 242 to the second end 244 of the sheath portion 240. The blade slot 252 may extend into the arm 250 from a top surface toward a bottom surface. The blade slot 252 may have, for example, a square or rectangular interior surface. The arm 250 may also include a cable channel 254 extending from the first end 242 to the second end 244 of the sheath portion 240. The cable channel 254 may extend into the arm 250 from the bottom surface toward the top surface. The cable channel 254 may have, for example, a round or curved interior surface. The blade slot 252 may extend farther into the arm 250 than the cable channel 254. The sheath portion 240 may also include an opening 256 at the first end 242 of the sheath portion 240. The opening 256 may be positioned at the first end 242 of the arm 250. The opening 256 may extend into the cable channel 254. The opening 256 may be, for example, larger than the cable channel 254. The opening 256 may be, for example, sized to receive the camera 308 and illumination element 310, such as LEDs.

As shown in FIGS. 12-13 and 20, the base 246 of the sheath portion 240 may be positioned within the recess 174 of the first housing 130 and the recess 226 of the second housing 190. The arm 250 extends through the blade holder slot 208. The fasteners 182 extend through the second housing 190, the base 246 of the sheath portion 240, and the first housing 130 to form the handpiece 120.

The cannula 270 may include a first end 272 opposite a second end 274 and a first side 276 opposite a second side 278, as shown in FIGS. 14-19. The first end 272 may be, for example, an insertion end. The second end 274 is configured or sized and shaped to engage the first end 112 of the handpiece 120. The cannula 270 may include a base 280 and an extension member 290 extending away from the base 280 to the first end 272. The base 280 is positioned at the second end 274 of the cannula 270. The base 280 also includes an exterior surface 294 that may engage or contact the patient's skin and an interior surface 296 that engages or contacts the coupled housings 130, 190. The base 280 includes a first arm 282 coupled to and extending away from a first side 276 of the base 280 and a second arm 284 coupled to and extending away from a second side 278 of the base 280. The first arm 282 and the second arm 284 may be positioned on opposite sides of the base 280. The arms 282, 284 may, for example, provide for a position to place the user's fingers during insertion, as well as assist with insertion and removal of the cannula 270 onto the coupled housings 130, 190. The arms 282, 284 may engage corresponding recesses 126, 128 in the first housing 130 and the second housing 190 of the handpiece 120. The first arm 282 may engage the recesses 126 and the second arm 284 may engage the recesses 128.

The base 280 may also include a protrusion 286 extending away from the exterior surface 294 of the base 280. The protrusion 286 may have a width the same width as the extension member 290. The base 280 may further include a blade slot 288 extending through the base 280 from the interior surface 296 to the exterior surface 294 and into the extension member 290. The first end 272 of the extension member 290 may include a leading end. The leading end 272 may be, for example, tapered, curved, or stepped from a bottom surface toward the top surface and from the first and second side surfaces to a top at the end of the first end 272. The leading end 272 may also be, for example, curved between a first or right side and a second or left side of the cannula 270. The extension member 290 may also include a channel 292 recessed into a top surface of the cannula 270. The channel 292 may extend into the cannula 270, for example, to give the cannula 270 a U-shaped cross-section along a majority of the length of the cannula 270. The cannula 270 may also have, for example, rounded edges along the length of the cannula 270. The channel 292 may extend from the base 280 to a position adjacent to the leading end 272. The channel 292 may open into the blade slot 288 to allow for insertion of the sheath portion 240 for deployment of the blade 320 for cutting tendinous, ligamentous, fibrous tissue, and the like. At least a portion of the cannula 270 may be, for example, clear, transparent, or opaque. For example, the cannula 270 may be made of a transparent plastic, such as, polycarbonate or a more rigid opaque polyacrylamide.

When at least a portion of the cannula is clear or see-through, the camera 308 may allow for visualization not only through the channel 292 of the cannula 270, but also through that clear portion of the extension member 290. A portion of the sheath portion 240 may be slidingly received within the channel 292 of the cannula 270.

The instrument 110 also includes a camera assembly 300 with a first end 302 and a second end 304. The camera assembly 300 includes a housing 306 at the first end 302. The housing 306 includes a camera or camera sensor 308 positioned at the first end of the housing 306. The housing 306 also includes at least one light member, illumination member, or LED 310. The at least one light member 310 may be, for example, a first light member 310 on a first side of the camera 308 and a second light member 310 on a second side of the camera 308. The camera 308 and at least one light member 310 are coupled to a cable 312 at the first end 302 of the camera assembly 300. The cable 312 is also coupled to electrical components 316, such as, circuit boards, bridge circuit boards, and the like, at the second end 304. The assembly 300 may also include a sheath 314 covering at least a portion of the cable 312. The sheath 314 may be positioned to extend from the housing 306 toward the second end 304. The sheath 314 may be positioned within the cable channel 254 of the sheath portion 240 to protect the cable 312 during use of the instrument 110. The sheath 314 may assist with protecting the cable 312 during translation of the cannula 270.

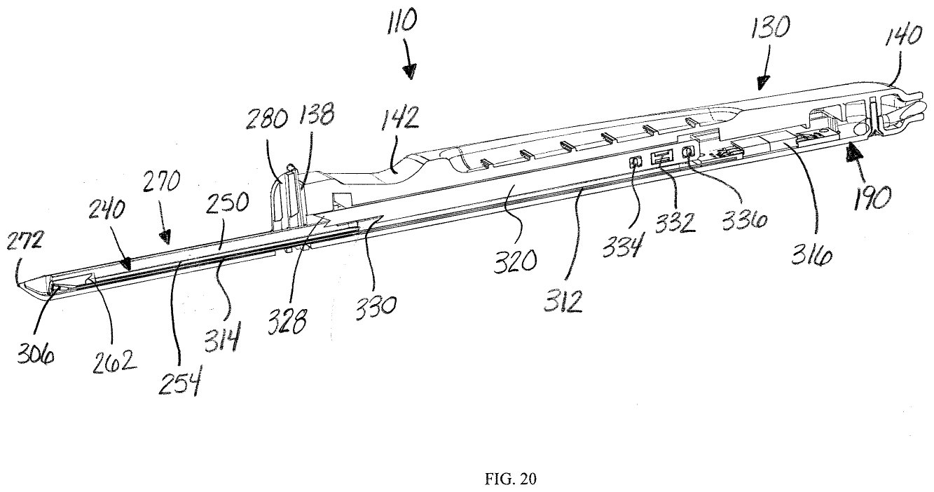

The instrument 110 further includes a blade 320 with a first end 322 opposite a second end 324. The blade 320 includes a body 326 with a first cutting element 328 at the first end 322 and a locking member 330 near the first end 322. The first cutting element 328 is positioned at the first end 322. The locking member 330 is positioned at a bottom of the body 326. Referring now to FIG. 20, the locking member 330 may engage at least a portion 262 of the sheath portion 240 to secure the locking member 330 in position. The at least a portion 262 may be a blade engagement portion or locking feature 262 with a corresponding shape to the locking member 330. The body 326 also includes a switch opening 332 near the second end 324. The switch opening 332 may extend through the body 326 from a first side to a second side. The body 326 further includes at least one attachment protrusion 334, 336. The at least one attachment protrusion 334, 336 includes a first attachment protrusion 334 positioned between the switch opening 332 and the first end 322 of the blade 320 and a second attachment protrusion 336 positioned between the switch opening 332 and the second end 324. Each attachment protrusion 334, 336 is positioned within a through hole positioned on either side of the switch opening 332. The blade 320 is configured or sized and shaped to be slidingly received within the blade channel 160 of the first housing 130 and the blade channel 212 of the second housing 190. The blade 320 translates within the blade channels 160, 212 and the blade slot 252 of the sheath portion 240.

The instrument 110 also includes a spring 340 with a first hook 342 at a first end and a second hook 344 at a second end. The first hook 342 is configured or sized and shaped to couple to the first attachment protrusion 334. The second hook 344 is configured or sized and shaped to couple to the second attachment protrusion 336.

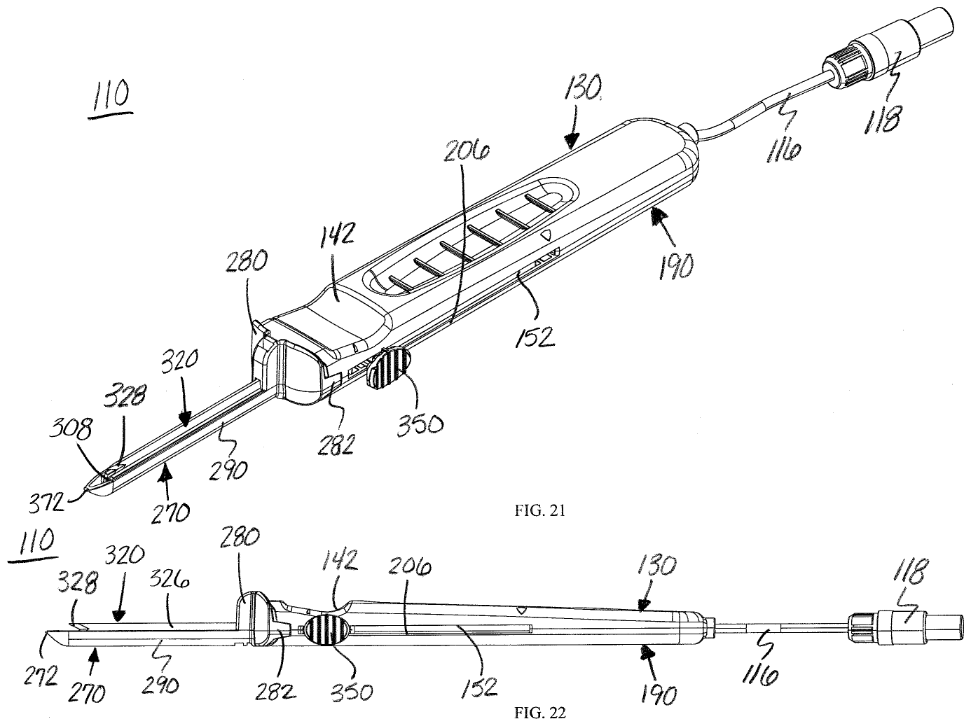

Further, the instrument 110 includes a switch or translating member 350 with a first end 352 and a second end 354. The switch 350 includes a shaft 352 and a button 364 coupled to a first end of the shaft 352. The shaft 352 includes a first protrusion 358 extending away from a top surface of the shaft 352 near the button 364. The shaft 352 also includes a second protrusion 360 extending away from a bottom surface of the shaft 352 near the button 364. The protrusions 358, 360 may be, for example, cylindrical in shape to allow for translation within the protrusion channels 168, 220 of the first and second housings 130, 190 respectively. The shaft 352 may also include a through hole 362 extending through the shaft 352 from a top surface to a bottom surface. The through hole 362 may receive a pin 370. The pin 370 may be, for example, removable from or integral with the shaft 352. The pin 370 may be, for example, cylindrical in shape to allow for translation within the pin channels 162, 214 of the first and second housings 130, 190, respectively. The button 364 may have, for example, a width and height larger than the width and height of the shaft 352. The button 364 may be, for example, coupled to the shaft 352 on a first side and have a curved shape on a second side. The curved side of the button 364 may also include, for example, ridges, grooves, or other texture to assist a user with translating the switch along the length of the coupled handpiece 120. The second end 354 of the shaft 352 may engage a portion of the spring 340. During use as pressure is applied to the button 364 of the switch 350, the spring 340 is stretched to allow for the switch 350 to move out of the extensions 164, 166, 170, 172, 216, 218, 222, 224 of the channels 162, 168, 214, 220. For example, when the blade 320 is in an undeployed position, the protrusions 358, 360 and pin 370 will be positioned within the extensions 164, 170, 216, 222 of the pin channels 162, 214 and the protrusion channels 168, 220. Then, pressure may be applied to the switch 350 and the spring 340 extended to move the protrusions 358, 360 and pin 370 into the pin channels 162, 214 and the protrusion channels 168, 220 to translate the blade 320 along the length of the instrument 110. When the blade 320 has reached a fully deployed position, as shown in FIGS. 21-22, the protrusions 358, 360 and pin 370 may be positioned within the extensions 166, 172, 218, 224 to secure the blade 320 in the deployed position for cutting. After cutting is complete, the blade 320 may be translated back to an undeployed position by reversing the translation of the blade 320 along the instrument 110.

A surgical method for using the videoscopic arthroscopic system 100 may include obtaining a release instrument 110. The release instrument 110 may then be coupled to the video unit, tablet, and/or power source by a cable 116. The cable 116 may transmit images of the surgical site to a video processor and/or video monitor, such as video unit 102. The video processor and monitor may be, for example, separate or combined, such as a combined processor and tablet display. Then, the surgeon may perform the initial procedure using their preferred technique. After the release instrument 110 is connected to a power source and video unit and the initial procedure is complete, the first end 112 of the release instrument 110 may be inserted into the surgical site of a patient. Specifically, the method includes inserting a distal portion of the instrument 110 into the carpal tunnel with the blade 320 in an undeployed position. For example, for an endoscopic carpal tunnel release procedure, the surgeon may introduce the instrument 110 into the carpal tunnel through a single limited incision in the wrist flexor crease. The camera assembly 300 may be used to visualize the path of the release instrument 110 as the surgeon inserts the instrument 110 into the patient to the position where tissue release is desired. For example, the deep side of the transverse carpal ligament (TCL) may be viewed under direct visualization at the tip of the instrument. After the instrument 110 is positioned at the location for tissue release, the surgeon may detach and retract the handle with the camera tip keeping the cannula stationary and providing a full-length view of the entire carpal canal. Thus, the handle and the camera tip may be removed from the clear cannula 270. Then, the blade 320 may be actuated by pressing in on the switch and advancing forward. The blade should be moved to a fully activated position by moving the switch 350 until the switch 350 extends back out of the handpiece 120. Once it is confirmed the switch 350 is in the hole then complete activation of the blade 320 is achieved. Next, the surgeon actuates the forward cutting blade 320 by pushing the blade 320 and camera 308 back into the cannula to cut the ligament, for example, transect the TCL. After the ligament is cut, the instrument may be withdrawn if complete release of the carpal tunnel was achieved. The instrument may be withdrawn by de-activating the blade 320 by pressing in on the switch 350 and sliding backwards. Once the blade 320 is fully deactivated, the switch 350 will extend back out of the handpiece 120. Alternatively, the instrument 110 may be withdrawn by removing the handpiece 120 with or without the cannula 270 with the blade 320 in the deployed position. After the instrument 110 is removed and disconnected, the instrument 110 may be disposed in a sharps container.

As may be recognized by those of ordinary skill in the art based on the teachings herein, numerous changes and modifications may be made to the above-described and other embodiments of the present disclosure without departing from the scope of the disclosure. The instruments, devices, and/or systems as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the instruments, devices, and systems may include more or fewer components or features than the embodiments as described and illustrated herein. For example, the components and features of the compression anchor systems described above may all be used interchangeably and in alternative combinations as would be modified or altered by one of skill in the art. Accordingly, this detailed description of the currently-preferred embodiments is to be taken in an illustrative, as opposed to limiting of the disclosure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

1. A release instrument, comprising:
a handpiece with a first end and a second end, wherein the handpiece comprises:
at least one housing extending from the second end of the handpiece towards the first end; and
a sheath portion extending from the at least one housing to the first end of the handpiece;
a translating member moveably coupled to the at least one housing;
a blade member moveably coupled to the translating member; and
a cannula with a first end and a second end, wherein the sheath portion of the handpiece is slidingly received within the cannula, and wherein the translating member moves the blade member from an undeployed position to a deployed position.

2. The release instrument of claim 1, wherein the at least one housing comprises:
a first housing; and
a second housing coupled to the first housing.

3. The release instrument of claim 2, wherein the first housing forms a top portion of the handpiece and wherein the second housing forms a bottom portion of the handpiece.

4. The release instrument of claim 2, wherein the first housing comprises:
a first channel on an interior surface of the first housing extending between at least a portion of the first end and the second end of the first housing;
a second channel on the interior surface of the first housing extending between at least a portion of the first end and the second end of the first housing and positioned between the first channel and a first side of the first housing; and
a third channel on the interior surface of the first housing extending between at least a portion of the first end and the second end of the first housing and positioned between the first channel and a second side of the first housing.

5. The release instrument of claim 4, wherein the second housing comprises:
a first channel on an interior surface of the second housing extending between at least a portion of the first end and the second end of the second housing;
a second channel on the interior surface of the second housing extending between at least a portion of the first end and the second end of the second housing and positioned between the first channel and a first side of the second housing; and
a third channel on the interior surface of the second housing extending between at least a portion of the first end and the second end of the second first housing and positioned between the first channel and a second side of the second housing.

6. The release instrument of claim 5, wherein the first channel of the first housing aligns with the first channel of the second housing when assembled, wherein the second channel of the first housing aligns with the second channel of the second housing when assembled, and wherein the third channel of the first housing aligns with the third channel of the second housing when assembled.

7. The release instrument of claim 6, wherein the translating member comprises:
a shaft with a first end and a second end; and
a button coupled to a first end of the shaft.

8. The release instrument of claim 7, wherein the shaft comprises:
a first protrusion extending away from a top surface of the shaft;
a second protrusion extending away from a bottom surface of the shaft; and
a through hole extending through the shaft from a top surface to a bottom surface near the second end of the shaft.

9. The release instrument of claim 8, wherein the through hole receives a pin coupled the shaft and including a first portion extending away from the top surface of the shaft and a second portion extending away from the bottom surface of the shaft.

10. The release instrument of claim 8, wherein the first protrusion slidingly engages the third channel of the first housing and wherein the second protrusion slidingly engages the third channel of the second housing.

11. The release instrument of claim 9, wherein the first portion of the pin slidingly engages the second channel of the first housing, and wherein the second portion of the pin slidingly engages the second channel of the second housing.

12. The release instrument of claim 9, further comprising:
a spring coupled to the blade member at each end and engaging the second end of the translating member.

13. The release instrument of claim 1, wherein the cannula is transparent.

14. A videoscopic arthroscopic system, comprising:
a release instrument including a blade and at least one camera sensor, wherein the blade and the at least one camera sensor translate together; and
a video unit coupled to the release instrument.

15. The system of claim 1, wherein the release instrument further comprises:
a handpiece with a first end and a second end, wherein the handpiece comprises:
at least one housing extending from the second end of the handpiece towards the first end; and
a sheath portion extending from the at least one housing to the first end of the handpiece; and
a translating member moveably coupled to the at least one housing;
wherein the blade is moveably coupled to the translating member; and
a cannula with a first end and a second end, wherein the sheath portion of the handpiece is slidingly received within the cannula, and wherein the translating member moves the blade from an undeployed position to a deployed position.

16. The system of claim 15, wherein the at least one housing comprises:
a first housing; and
a second housing coupled to the first housing.

17. The system of claim 16, wherein the translating member comprises:
a shaft with a first end and a second end and positioned within the coupled first housing and the second housing; and
a button coupled to a first end of the shaft.

18. The system of claim 17, wherein at least a portion of the shaft of the translating member extends through an opening formed between the coupled first housing and second housing to position the button on an exterior side of the first housing and the second housing.

19. The system of claim 15, wherein the blade translates through a blade slot extending into a top surface of the sheath portion; and wherein the at least one camera sensor is coupled to a cable positioned within a cable channel extending into a bottom surface of the sheath portion.

20. A surgical method for releasing tissue, comprises:
obtaining a videoscopic arthroscopic system;
creating an incision in a patient;
inserting a release instrument into the incision;
using an integrated camera of the release instrument to position a cannula of the release instrument adjacent to a tissue to be cut;
deploying the blade from the release instrument;
translating a handpiece of the release instrument within the cannula to translate the blade to cut the tissue to be cut;
removing the release instrument; and
completing the procedure.
